(19) United States
(12) Patent Application Publication
(10) Pub. No.: US 2026/0044956 A1
(43) Pub. Date: Feb. 12, 2026

(54) A METHOD AND SYSTEM FOR STAGING DIABETIC KIDNEY DISEASE USING DEEP LEARNING

(71) Applicants: CARL ZEISS MEDITEC, INC., Dublin, CA (US); CARL ZEISS MEDITEC AG, Jena (DE)

(72) Inventors: Raghu Prasad, Bangalore (IN); Anvesh Vankayala, Bangalore (IN); Ankit Chavhan, Bangalore (IN); Mansi Gupta, Bangalore (IN); Ganesh Babu T C, Bangalore (IN)

(73) Assignees: CARL ZEISS MEDITEC, INC., Dublin, CA (US); CARL ZEISS MEDITEC AG, Jena, TH (DE)

(21) Appl. No.: 19/140,707

(22) PCT Filed: Jan. 4, 2024
(86) PCT No.: PCT/EP2024/050125

§ 371 Date: Jun. 18, 2025

(30) Foreign Application Priority Data
May 1, 2023 (IN) 202311001136

(51) Int. Cl. G06T 7/00; A61B 3/00; A61B 3/10; A61B 3/12; G06T 5/20; G06T 5/70

(52) U.S. Cl. CPC G06T 7/0012; A61B 3/0025; A61B 3/0041; A61B 3/102; A61B 3/12; G06T 5/20; G06T 5/70; A61B 2576/02; G06T 2207/10004; G06T 2207/10048; G06T 2207/10101; G06T 2207/20081; G06T 2207/20172; G06T 2207/30041; G06T 2207/30104

(57) ABSTRACT

Embodiments herein disclose a method and system for staging diabetic kidney disease using deep learning techniques. An image capturing unit captures a set of ophthalmic images of a user. The ophthalmic images set undergoes pre-processing before being fed to a first deep learning module. The first deep learning module extracts pathological data indicative of vascular abnormalities from the pre-processed set of ophthalmic images. The first deep learning module quantifies the extracted pathological data, and maps them to a stage of diabetic retinopathy and urine protein levels. A second deep learning module receives as input the quantified pathological data, the mapped diabetic retinopathy stage and urine protein levels, and clinical and demographic parameters. Based on this input, the second deep learning module predicts a stage of diabetic kidney disease.

### CROSS-REFERENCE TO RELATED APPLICATIONS

This application claims the benefit or and priority to Indian Provisional Application No. 20/2311001136, the entire disclosure of which is hereby incorporated by reference in its entirety.

### TECHNICAL FIELD

Embodiments of the present invention generally relate to deep learning techniques for predicting diseases, and more particularly relates to deep learning techniques for detecting a stage of diabetic kidney disease.

### BACKGROUND

Diabetes (also referred to as Diabetes Mellitus) is characterized by group of metabolic disorders that share common phenotype of high blood sugar concentration. The prevalence of diabetes in adults has been increasing over recent decades globally. An estimated 415 million people were found to be affected by diabetes in 2015 and the International Diabetes Federation (IDF) predicts an increase to 642 million by 2040 with the greatest increase expected in Asia, in particular, India and China. This increase in prevalence of diabetes, along with the aging of population, will inevitably lead to an increase in microvascular complications like Diabetic Retinopathy (DR). DR, a leading cause of preventable blindness, has been shown to increase the risk of all-cause and cardiovascular death. DR and Diabetic Kidney Disease (DKD) (also known as Diabetic Nephropathy (DN)) share similar etiopathogenetic mechanisms, leading to comparable metabolic consequences DKD is a leading cause of end-stage renal disease (ESRD) eventually leading to a significant rise in the rate of morbidity and mortality. Furthermore, the risk of DKD progression increases with the risk of DR severity.

DKD is often unrecognized in the initial stages due to lack of routine screening for microalbuminuria (a range of urine protein levels), especially in low resource settings. The current management for ESRD includes either frequent dialysis or renal replacement therapy which means a significant cost implication and compromised quality of life for the patients and increased burden on the healthcare systems.

Though there exists good amount of evidence of detecting kidney disorders from diabetic fundus images (i.e., retinal images through which DR is detected), none of the past studies have concentrated predicting the stage of the kidney disease. None of the research works have analysed the impact of the presence of DR and non-DR patterns for staging the kidney disease. Further, none of the earlier prior arts have identified Microaneurysm(s), Dot/Blot hemorrhages, Hard Exudates, Cotton wool spots, Intraretinal hemorrhages, Venous beading, Intraretinal microvascular abnormalities, and infections to classify the fundus images into proliferative, non-proliferative DR. In particular, for proliferative DR, none of the prior arts have identified Microaneurysm(s), Dot/Blot hemorrhages, Hard Exudates, Cotton wool spots, Intraretinal hemorrhages, Venous beading, Intraretinal microvascular abnormalities, Neovascularization, Vitreous/Preretinal hemorrhage to classify the fundus images. So, Neovascularization, Vitreous/Preretinal hemorrhage are deciding pathologies to classify proliferative and non-proliferative DR stages. Further, none of the earlier prior art discloses techniques to determine the referable criteria to nephrologist.

### SUMMARY OF THE INVENTION

These and other problems are generally solved or circumvented, and technical advantages are generally achieved, by advantageous embodiments of the present disclosure.

The following presents a simplified summary of the subject matter in order to provide a basic understanding of some of the aspects of subject matter embodiments. This summary is not an extensive overview of the subject matter. It is not intended to identify key/critical elements of the embodiments or to delineate the scope of the subject matter. Its sole purpose is to present some concepts of the subject matter in a simplified form as a prelude to the more detailed description that is presented later.

In a first aspect, a system is provided. The system comprises an image capturing unit configured to capture a set of ophthalmic images of a person. The system further comprises an image processing unit configured to process the set of ophthalmic images as obtained from the image capturing unit. The image processing unit comprises a pre-processing module that is configured to separately pre-process the set of ophthalmic images. The image processing unit further comprises a first deep learning module configured to extract pathological data indicative of vascular abnormalities from the pre-processed set of ophthalmic images, quantify the extracted pathological data based on the vascular abnormalities, and map the quantified pathological data to a stage of diabetic retinopathy and urine protein levels. The image processing unit further comprises a second deep learning module configured to receive clinical and demographic parameters, and receive from the first deep learning module the quantified pathological data, the stage of diabetic retinopathy, and the urine protein levels. The second deep learning module is configured to predict a stage of diabetic kidney disease based on: the quantified pathological data, the stage of diabetic retinopathy, the urine protein levels, and the clinical and demographic parameters.

In a second aspect, a method is provided. The method comprises capturing, by an image capturing unit, a set of ophthalmic images of a person. The method comprises processing, by an image processing unit, the set of ophthalmic images as obtained from the image capturing unit. The processing comprises pre-processing, by a pre-processing module, the set of ophthalmic images. The processing comprises extracting, by a first deep learning module, pathological data indicative of vascular abnormalities from the pre-processed set of ophthalmic images. The processing comprises quantifying, by the first deep learning module, the extracted pathological data based on the vascular abnormalities. The processing comprises mapping, by the first deep learning module, the quantified pathological data to a stage of diabetic retinopathy and urine protein levels. The processing comprises receiving, by a second deep learning module, clinical and demographic parameters, and the quantified pathological data, the stage of diabetic retinopathy, and the urine protein levels, from the deep learning module. The processing comprises predicting, by the second deep learning module, a stage of diabetic kidney disease based on the quantified pathological data, the stage of diabetic retinopathy, the urine protein levels, and the clinical and demographic parameters.

In a third aspect, a computer-readable medium is provided. The computer-readable medium comprises instructions that, when executed by at least one processor in a computer system, cause the computer system to perform the method of the second aspect.

### BRIEF DESCRIPTION OF THE ACCOMPANYING DRAWINGS

The following drawings are illustrative of particular examples for enabling systems and methods of the present disclosure, are descriptive of some of the methods and mechanism, and are not intended to limit the scope of the invention. The drawings are not to scale (unless so stated) and are intended for use in conjunction with the explanations in the following detailed description.

FIG. 1 illustrates a relationship between vascular abnormalities in the eye and the vascular abnormalities in the kidney for an individual having diabetes;

FIG. 2 illustrates a process flow for detecting stage of Diabetic Kidney Disease (DKD), in accordance with an example embodiment of the present disclosure;

FIG. 3 illustrates a process flow for a two-step multilabel image model and a clinical model, in accordance with an example embodiment of the present disclosure;

FIG. 4 illustrates a work flow of the two-step multilabel image model and the clinical model for detecting the stage of Diabetic Retinopathy (DR) and Diabetic Kidney Disease (DKD), respectively, in accordance with an example embodiment of the present disclosure;

FIG. 5 illustrates an architecture of a two-step multilabel image model, in accordance with an example embodiment of the present disclosure;

FIG. 6 illustrates the working of the clinical model, in accordance with an example embodiment of the present disclosure;

FIG. 7 illustrates the process flow for detecting stage of DKD by the clinical model using the output of the two-step multilabel image model, in accordance with an example embodiment of the present disclosure;

FIG. 8 illustrates a process flow for performing retinal and renal assessment in an ophthalmology clinic and a nephrology clinic, respectively, in accordance with an example embodiment of the present disclosure;

FIGS. 9A and 9B illustrate various parameters and data statistics regarding the multilabel image model and the clinical model, in accordance with an example embodiment of the present disclosure;

FIGS. 10A and 10B illustrate a confusion matrix for the two-step multilabel image model and the clinical model, in accordance with an example embodiment of the present disclosure;

FIGS. 11A and 11B illustrate a graph depicting the performance of the two-step multilabel image model and the clinical model, respectively, in accordance with an example embodiment of the present disclosure;

FIGS. 12A and 12B illustrate pie charts representing a percentage breakdown of DR stages correlating to early stage DKD or advanced stage DKD, respectively, in accordance with an example embodiment of the present disclosure;

FIG. 13 illustrates a system for implementing the example embodiments of the present disclosure; and

FIG. 14 illustrates a method for detecting stage of DKD based on stage of DR, in accordance with an example embodiment of the present disclosure.

Persons skilled in the art will appreciate that elements in the figures are illustrated for simplicity and clarity and may represent both hardware and software components of the system. Further, the dimensions of some of the elements in the figure may be exaggerated relative to other elements to help to improve understanding of various exemplary embodiments of the present disclosure. Throughout the drawings, it should be noted that like reference numbers are used to depict the same or similar elements, features, and structures.

### DETAILED DESCRIPTION

Exemplary embodiments now will be described. The disclosure may, however, be embodied in many different forms and should not be construed as limited to the embodiments set forth herein; rather, these embodiments are provided so that this disclosure will be thorough and complete, and will fully convey its scope to those skilled in the art. The terminology used in the detailed description of the particular exemplary embodiments illustrated in the accompanying drawings is not intended to be limiting. In the drawings, like numbers refer to like elements.

The specification may refer to “an”, “one” or “some” embodiment(s) in several locations. This does not necessarily imply that each such reference is to the same embodiment(s), or that the feature only applies to a single embodiment. Single features of different embodiments may also be combined to provide other embodiments. As used herein, the singular forms “a”, “an” and “the” are intended to include the plural forms as well, unless expressly stated otherwise. It will be further understood that the terms “includes”, “comprises”, “including” and/or “comprising” when used in this specification, specify the presence of stated features, integers, steps, operations, elements, and/or components, but do not preclude the presence or addition of one or more other features, integers, steps, operations, elements, components, and/or groups thereof. As used herein, whenever the phrase “at least one of the following” precedes a list of elements, wherein the elements are joined by “and” or “or”, it means that at least any one of the elements or at least all the elements are present. As used herein, the term “and/or” includes any and all combinations and arrangements of one or more of the associated listed items.

Conditional language-such as “can” or “may”-among others, unless specifically stated otherwise, or otherwise understood within the context as used, is generally intended to convey that certain embodiments could include, while other embodiments may not include certain features, elements, and/or steps. Thus, such conditional language is not generally intended to imply that features, elements, and/or steps are in any way required for one or more embodiments. It will be understood that when an element is referred to as being “connected” or “coupled” to another element, it can be directly connected or coupled to the other element or intervening elements may be present. Furthermore, “connected” or “coupled” as used herein may include wirelessly connected or coupled.

Unless otherwise defined, all terms (including technical and scientific terms) used herein have the same meaning as commonly understood by one of ordinary skill in the art to which this invention pertains. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

The figures depict a simplified structure only showing some elements and functional entities, all being logical units whose implementation may differ from what is shown. The connections shown are logical connections; the actual physical connections may be different. In addition, all logical units described and depicted in the figures include the software and/or hardware components required for the unit to function. Further, each unit may comprise within itself one or more components, which are implicitly understood. These components may be operatively coupled to each other and be configured to communicate with each other to perform the function of the said unit.

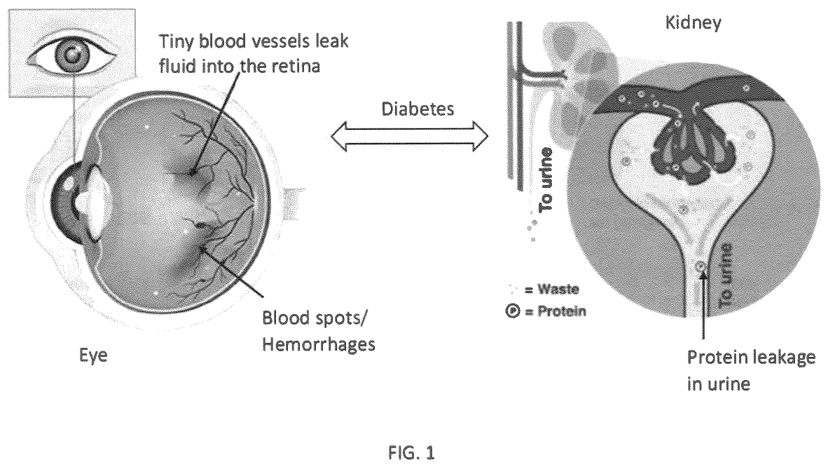

FIG. 1 illustrates a relationship between vascular abnormalities in the eye and the vascular abnormalities in the kidney of an individual having diabetes. Diabetic Retinopathy (DR) and Diabetic Kidney Disease (DKD) are comorbidities of diabetes and hence both the diseases have similar metabolic consequences. Diabetes affects the vasculature of eye and kidney in a similar way. As shown in FIG. 1, in the eye, diabetes can cause vascular damage leading to various types of vascular abnormalities, which can result in leakage of blood (seen as bleeding spots). Similarly, vascular damage in the kidney can lead to leakage of protein in the urine. The extent of vascular damage in the eye is similar to the extent of vascular damage in the kidney, as the vascular defects due to diabetes occurs similarly in kidneys, thereby affecting its protein filtration function.

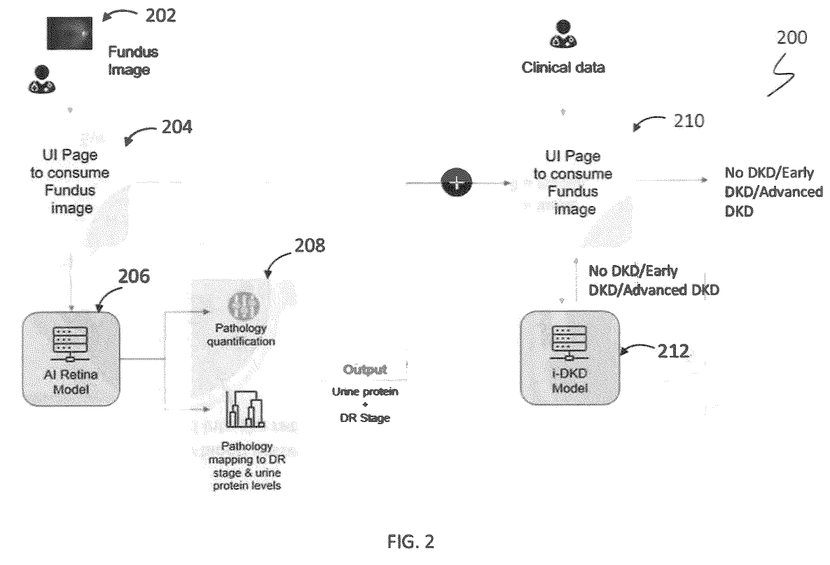

FIG. 2 illustrates a process flow 200 for detecting a stage of Diabetic Kidney Disease (DKD), according to an example embodiment of the present disclosure. At process block 102, a fundus or optical coherence tomography (OCT) image(s) is captured. The fundus image can be captured by an image capturing unit such as, but not limited to, a fundus camera, whereas the OCT image can be captured by an imaging capturing unit such as, but not limited to, an OCT machine. The description of FIG. 2 will be explained in the context of a fundus image as the captured image, however, this is to be construed as non-limiting as an OCT image can be used in the alternative. The fundus images may be a two-dimensional (2D) or a three-dimensional (3D) OCT cube representation of retinal images. In one embodiment, the fundus image may be an infrared image, and in another embodiment, the fundus image may be an autofluorescence image. The image data may be obtained in any one format among, but not limited to, JPG, PNG, DCM (DICOM), BMP, GIF, and TIFF.

At process block 204, the captured fundus image is fed into a computing device having a display on which a user interface is provided. The computing device may receive the captured fundus image either directly or indirectly from the image capturing device via wireless communication means such as Bluetooth, near field communication, Wi-Fi etc. The user interface may have an uploading option via which the fundus image may be uploaded. The computing device may be present with a doctor or a medical technician. The doctor or the medical technician captures the retinal fundus image of a diabetic patient who visits them.

At process block 206, the retinal fundus image is fed into an artificial intelligence (AI) retina model (i.e., the two-step multilabel image model) stored in the computing device. The AI retina model is used to take as input the fundus image and predict DR stages and urine protein levels based on the fundus image. In an example embodiment, the AI retina model can extract a total of 29 pathologies from the fundus images using deep learning techniques. At the training stage, the AI retina model is used to take as input a plurality of retinal fundus images labelled with pathologies (as part of a training dataset) and apply deep learning techniques to process and extract pathologies from the retina fundus images.

At process block 208, AI retina model quantifies the extracted pathological data in the captured fundus image. In an example embodiment, the pathological data is quantified as no abnormalities, microaneurysms, dot or blot hemorrhages, hard exudates, cotton wool spots, intraretinal hemorrhages, venous beading, intraretinal microvascular abnormalities, neovascularization, and/or vitreous or preretinal hemorrhage. The quantified pathological data is then mapped, using a DR mapper, to a DR stage and urine protein levels. In an example embodiment, the mapping of the quantified pathological data to a DR stage is done as per Table A.

TABLE A

| DR Stage | Pathologies |
|---|---|
| No DR | No abnormalities |
| Mild non | Microaneurysms |
| proliferative DR | |
| Moderate non- | Microaneurysm(s), Dot/Blot hemorrhages, Hard |
| proliferative DR | Exudates, Cotton wool spots |
| Severe non- | Microaneurysm(s), Dot/Blot hemorrhages, Hard |
| proliferative DR | Exudates, Cotton wool spots, Intraretinal |
| | hemorrhages, Venous beading, Intraretinal |
| | microvascular abnormalities |
| Proliferative DR | Microaneurysm(s), Dot/Blot hemorrhages, Hard |
| | Exudates, Cotton wool spots, Intraretinal |
| | hemorrhages, Venous beading, Intraretinal |
| | microvascular abnormalities, Neovascularization, |
| | Vitreous/Preretinal hemorrhage |

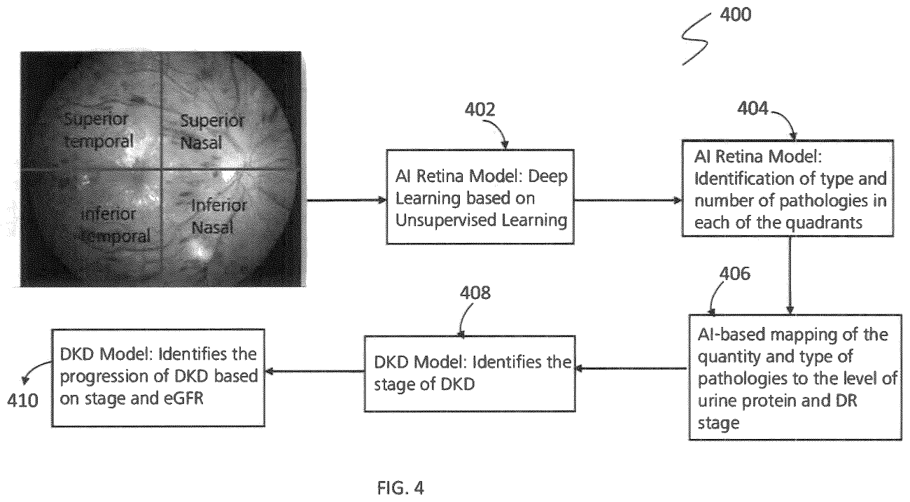

Depending on the type and number of pathologies, the DR stage of a diabetic patient can be predicted. Table A illustrates the following DR stages: no DR, mild non-proliferative DR, moderate non-proliferative DR, severe non-proliferative DR, and proliferative DR, in their increasing order of health risk. Hence, by quantifying the pathological data, the DR stage of the diabetic patient can be. Similarly, the quantified pathological data can also be used to predict urine protein levels by mapping to a standard reference range for urine protein levels, as normal (<30 mg/dL), microalbuminuria (30-300 mg/dL), and macroalbuminuria (>300 mg/dL). The mapping to the urine protein levels may be based on the quantity and type of vascular damage identified (by using deep learning techniques) in the four quadrants of a retinal fundus image (see FIG. 4).

In one example embodiment, the computing device storing the AI retina model can also store the clinical model. In another example embodiment, there may be separate computing devices to store the AI retina model and the DKD model (i.e., the clinical model). At process block 210, the computing device storing the clinical model receives the output of the AI retina model, i.e., the DR stage and the urine protein levels. Additionally, in some embodiments, the computing device also receives clinical data relating to a patient. The clinical data can include data about a patient's history of diabetes, whether the patient has other comorbidities etc. The computing device storing the DKD model can have a UI page on its display, wherein the UI page includes multiple input fields through which the DR stage, urine protein levels, and the clinical data can be manually entered. The captured fundus image at process block 202 can also be uploaded via an upload function on the UI page of the computing device.

At process block 212, the DKD model takes as input the DR stage, urine protein levels, and the clinical data, to output a predicted stage of the DKD. The predicted stage of DKD can be used to classify the seriousness of the DKD, as shown in Table B below.

TABLE B

| DKD Stage | Classification |
|---|---|
| 0 | No DKD |
| 1, 2, or 3 | Early stage |
| 4 or 5 | Advanced Stage |

The predicted stage of DKD and/or its classification may be displayed on the UI page of the computing device storing the DKD model.

In one embodiment, the DKD model performs binary classification to classify the DKD stage as early stage or advanced stage. In another embodiment, the DKD model performs multilabel classification to classify the DKD stage as no DKD, early DKD, or advanced DKD (as shown in Table B).

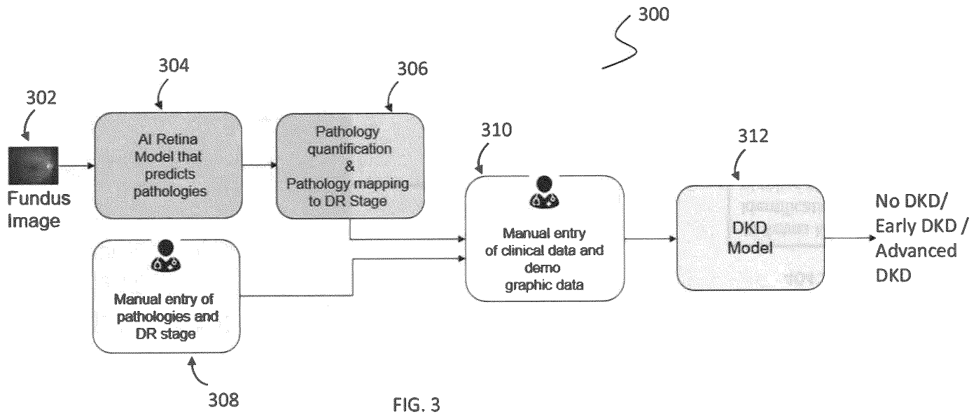

FIG. 3 illustrates a simple process flow 300 for the AI retina model (i.e., the two-step multilabel image model) and the DKD model (i.e., clinical model). At process block 302, the fundus image of a diabetic patient is captured and fed to the AI retina model. At process block 304, the AI retina model receives the fundus image for predicting the DR stage and urine protein levels of the diabetic patient based on the fundus image. More particularly, at process block 304, the AI retina model extracts pathological data from the captured fundus image, which can be used for predicting the DR stage and urine protein levels. At step 306, the AI retina model quantifies the extracted pathological data into zero or more pathologies (i.e., classifying the fundus image into zero or more pathologies). Based on the quantified pathological data, a DR mapper is responsible for mapping the quantified pathological data to a DR stage. The DR mapper can be a DR stage Regex rule parser that uses deep learning techniques to perform the mapping as per Table A. The quantified pathological data can also be used for mapping to urine protein levels.

At process block 308, the output of the AI retina model, i.e., the DR stage and the urine protein levels are manually entered as inputs to the DKD model. In some embodiments, the output of the AI retina model may be fed directly to the DKD model, i.e., there may not be a need for a manual entry of this data (process block 308). At process block 310, clinical and demographic data about the diabetic patient may be manually entered as input to the DKD model. Examples of clinical data include other comorbidities, duration of diabetes, history of hypertension etc. Demographic data can include age and gender.

At process block 312, the DKD model is able to predict the stage of DKD within the diabetic patient based on the input received at process blocks 306/308 and 310. The stage of DKD can be classified as no DKD, early DKD, or advanced DKD.

FIG. 4 illustrates a work flow of the two-step multilabel image model and the clinical model for detecting the stage of Diabetic Retinopathy (DR) and Diabetic Kidney Disease (DKD) based on four quadrants of a fundus image, according to an example embodiment disclosed herein.

The fundus image can be divided into four quadrants: superior temporal, superior nasal, inferior temporal, and inferior nasal. At process block 402, the AI retina model is trained to identify the type and number of pathologies using unsupervised learning. In some embodiments, the AI retina model may be trained via supervised learning using a labelled training dataset. At process block 404, once the AI retina model is trained, it identifies the type and number of pathologies in each quadrant. At process block 406, the quantity and type of pathologies are mapped to a DR stage and urine protein levels via AI-based techniques. At process block 408, based on the DR stage and the urine protein levels, the DKD model identifies the stage of DKD. At process block 410, based on the DKD stage and estimated glomerular filtration rate (eGFR), the progression of DKD is identified. The eGFR may be estimated based on a patient's blood samples.

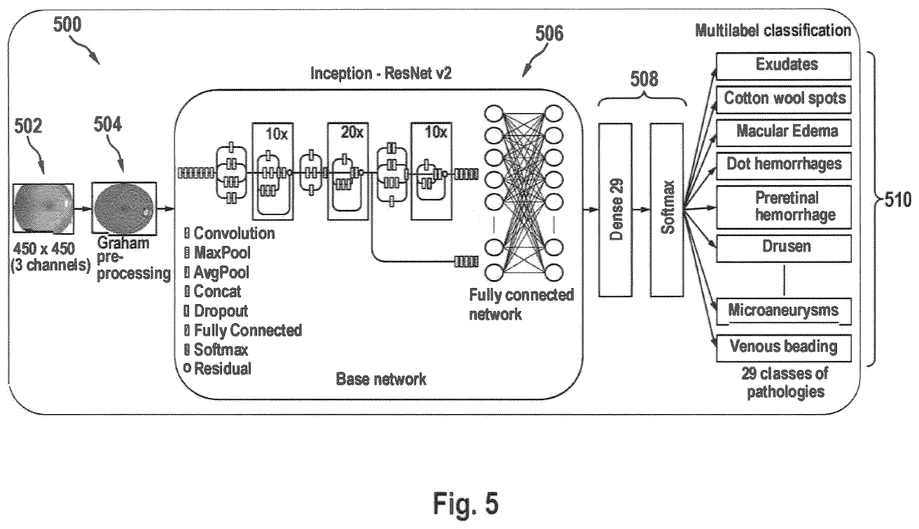

FIG. 5 illustrates an architecture 500 of the two-step multilabel image model (i.e., the AI retina model), according to an embodiment disclosed herein. The multilabel image model can extract the pathological data representative of vascular abnormalities patterns from a fundus image. The extracted pathological data is then quantified, and mapped to a DR stage and urine protein levels. The extracted pathological data and the DR stage are used to model the clinical model (i.e., the DKD model).

### Training of Two-Step Multilabel Image Model

In order for the two-step multilabel image model to perform its aforementioned functions, the multilabel image model may undergo training. In an example embodiment, the multilabel image model may be trained using fundus data with 29 classes of pathologies. In an example embodiment, the dataset used to model the multilabel image model includes 133273 samples for training set and 14779 samples for testing set. The total number of color fundus images may be labeled with zero to more pathologies and the DR stage by a clinician. The training set can include retinal fundus images with zero or more pathologies, wherein in the case of zero pathologies, it can be determined that there is no DR. This can help in training the two-step multilabel image model to associate a DR stage with the quantified pathological data.

In an embodiment, the multilabel image model uses InceptionRestNetV2 as the base model. Prior to processing the fundus image, techniques such as Gaussian blur can be used to smooth the images first and then Ben Graham is used for pre-processing of fundus images. In Graham both scaling and circular crop can be added.

In deep learning, a convolutional neural network (CNN) may be the category of deep neural networks, that are most likely applied to capture spatial information in visual imaging. As previously mentioned, the multilabel image model has InceptionResNetV2 as a base model. InceptionRestNetV2 is a CNN based pre-trained net with 164 layers depth and trained with ImageNet database images. The multilabel image model also includes three custom dense layers. By setting the top parameter to false, the last layer of the model is subtracted, enabling the customized dense layer to be used for training. This is essentially transfer learning, extracting the features of base model and using them to train the fundus data by adding custom dense layers. By using transfer learning, there is an efficient utilization of resources, as there is now avoidance of utilizing resources for training a model from scratch. The three custom dense layers are added with 256, 128, and 29 neurons in each layer. Lastly, Softmax is used as last layer activation, as there are zero to multiple pathologies for each image. Cross-entropy is used as training loss. Classes are weighted based on class frequency, and empty and highly under-represented classes are given with fixed small weight.

The output of the multilabel model is a classification of the input fundus image into zero or more pathologies, which is basically multi-label classification.

Thus, as shown in an example embodiment according to FIG. 5, at process step 502, a fundus image of 450×450×3 is received. As mentioned above, the fundus image may be retinal fundus image, i.e., fundus image relating to ophthalmic data of a patient. At process step 504, Graham pre-processing is performed on the received fundus image to pre-process fundus image. In some embodiments, Gaussian blur is applied on the fundus image to smoothen it prior to performing the Graham pre-processing.

At process step 506, the pre-processed images are fed to the Inception-ResNet v2 architecture. The Inception-ResNet v2 is a convolution neural network that is trained on more than a million images from ImageNet Database. This neural network is used to classify images into multiple categories using deep learning techniques. The neural network comprises a base network and a fully connected network.

At process step 508, three custom dense layers are added. In one embodiment, the three layers are added with 256, 128 and 29 neurons in each layer. Further, at process step 508, SoftMax is used as last layer activation, as zero to multiple pathologies for each image can be used. The dense layer is used to define relationship between values of the data in which the model is working. Further, the SoftMax is used for final classification of the data.

At process step 510, the multilabel classification is performed. This classifies the fundus image into one or more pathological classes (for example up to 29 classes). Some of the multilabel classification include, but not limited to, classification of exudates, cotton wool spots, macular edema, dot hemorrhages, preretinal hemorrhages, drusen, microaneurysms, and venous beading. In some embodiments, the classification can also include a class of “no vascular abnormalities.” In an embodiment, the multilabel image model can accept input such as urine protein levels, urine creatinine levels, and protein to creatine ratio for predicting a DR stage.

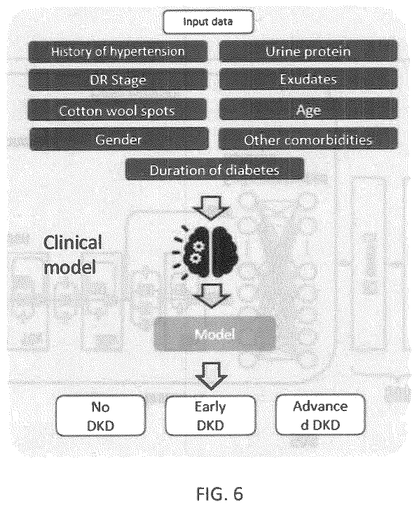

FIG. 6 illustrates the working of the clinical model, according to an example embodiment disclosed herein, is shown. In the clinical model, the feature selection is performed using the statistical tests, machine learning forward feature selection technique, machine learning experiments, and clinical expert opinion. The features may be categorized into different categories such as continuous/categorical or new. A table showing of the categorization of features is shown below:

| Features | Categorization |
|---|---|
| History of hypertension | Categorical |
| Urine protein | Categorical |
| DR Stages | Categorical |
| Cotton wool spots | Categorical |
| Exudates | Categorical |
| Age | Continuous |
| Gender | Categorical |
| Other comorbidities | Categorical |
| Duration of diabetes | Continuous |

As shown above, while the features-history of hypertension, Urine protein, DR stage, Cotton wool spots, Exudates Gender and age are categorical variables, the features-age and duration of diabetes are continuing features. A binary classifier is a meta estimator that fits several decision tree classifiers on various sub-samples of dataset. In one embodiment, the binary classifier uses averaging to improve the predictive accuracy and control over-fitting. Controlling of overfitting is important such that the accuracy of prediction can be improved. The sub-sample size is controlled with max samples otherwise whole data set is used. Max features and tree height are used to control over-fitting. In one embodiment, the final ensemble model used may be Random Forest. The clinical model can receive inputs such as the DR stage, urine protein levels range, and perform a 2-class classification. This can be Inception ResNet based 2-class classification algorithm, that classifies the stage of the DKD as early or advanced.

As shown in FIG. 3, the selected features can be manually input into the clinical model. In some embodiments, instead of binary classification (i.e., early DKD or advanced DKD), the clinical model can perform multilabel classification (no DKD, early DKD, advanced DKD, or late stage DKD). In another embodiment, the DKD model can predict the accumulation and release of proteins from the kidney.

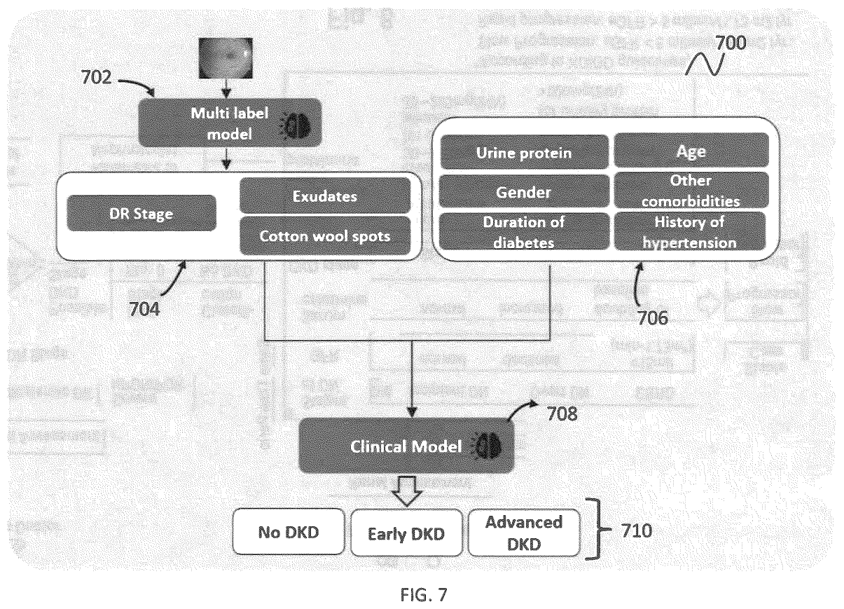

FIG. 7 illustrates a process flow 700 for detecting stage of DKD using two-step multilabel image model and the clinical model, according to an embodiment disclosed herein.

As per process flow 702, the multilabel image model receives the fundus image and performs automated feature extraction of the fundus image to extract the pathological data from it. At process flow 704, the multilabel image model performs multilabel classification to quantify the extracted pathological data into one or more pathology classes (e.g., exudates and/or cotton wool spots). At step 704, the quantified pathological data, is mapped to a DR stage and urine protein levels. The quantified pathological data can include at least one of: exudates, cotton wool spots, microaneurysms, and/or venous beading. The DR stage may be computed by a DR stage Regex rule parser that maps the quantified pathological data to a DR stage. At process flow 706, the quantified pathological data is mapped to urine protein levels. At process flow 706, the various clinical and demographic parameters of the diabetic individual are determined.

As per process flow 708, the clinical model gets the manual entry of ophthalmic related features like DR stage, quantified pathological data (e.g., exudates and cotton wool spots), urine protein levels, and clinical and demographic data (e.g., age, gender, other comorbidities, duration of diabetes, history of hypertension). Using this input, at process flow 710, the clinical model predicts the stage of DKD. In some embodiments, the DKD model uses binary classification to output either early DKD or advanced DKD. In other embodiments (as shown in FIG. 7), the DKD model uses multilabel classification to output no DKD, early DKD, or advanced DKD.

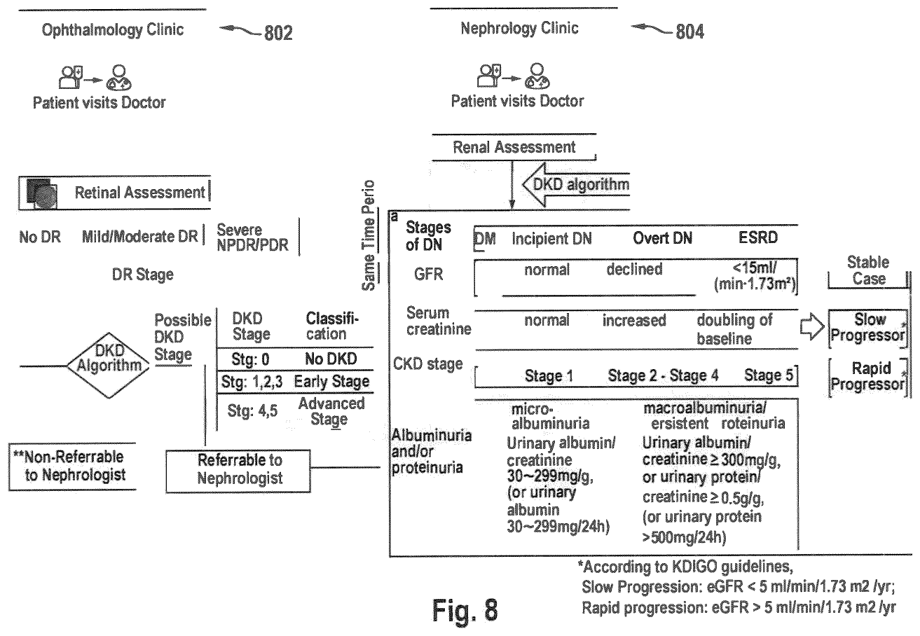

FIG. 8 illustrates a process flow 802 for a retinal assessment and a process flow 804 for a renal assessment in an ophthalmology clinic and a nephrology clinic, respectively, according to an embodiment disclosed herein. In ophthalmology clinic, when a diabetic patient visits a doctor present in the clinic, the doctor performs retinal assessment of the patient. From the retinal assessment, stage of the DR can determined. The stages of the DR can be classified as no DR, Mild/Moderate DR and Severe Non Proliferative DR/Proliferative DR. The retinal assessment can be performed using the AI retina model (i.e., the multilabel image model) or by a doctor. The DR stage is then fed into the DKD model. The DKD model predicts the stage of the DKD, which can be classified as early stage or advanced stage. Based on the stage of the DKD, the doctor at ophthalmology clinic can decide whether it constitutes as referable criteria for referring the patient to a nephrologist. The different stages of DKD and their respective classification is illustrated below:

| Stage | Classification |
|---|---|
| Stage 0 | No DKD |
| Stage 1, 2 or 3 | Early Stage |
| Stage 4 or 5 | Advanced Stage |

In some embodiments, the DKD model can predict the DKD stage to be 0, which is classified as “no DKD.” If the patient is referred to a nephrologist, then the patient visits a nephrology clinic. At the nephrology clinic, the doctor takes the retinal fundus images of the patient. The patient who visits the nephrology clinic can be assumed to have diabetic retinopathy (DR) disease. The renal assessment performed on the diabetic patient includes applying the DKD model. The output of the renal assessment includes a plurality of data such as the stage of DKD, the glomerular filtration rate, serum creatinine level, chronic kidney disease (CKD) stage, and albuminuria levels. The DKD algorithm detects stage of the DKD as early or advanced. The CKD stage can be classified as stable case, slow progressor, or rapid progressor.

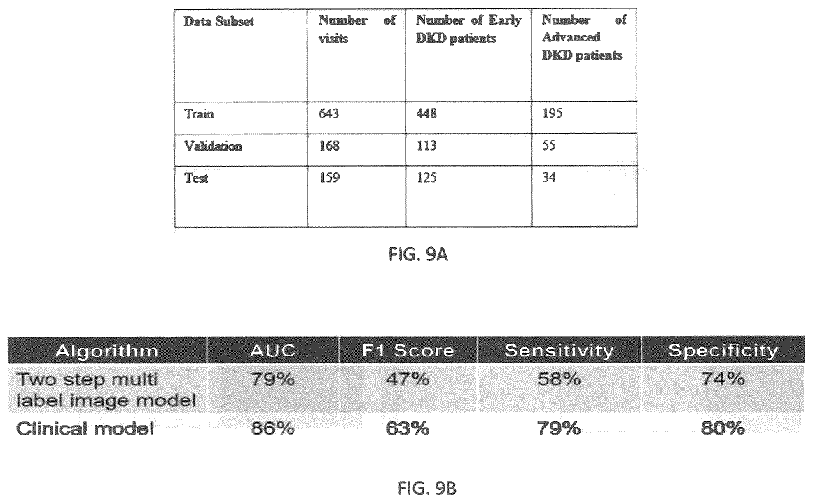

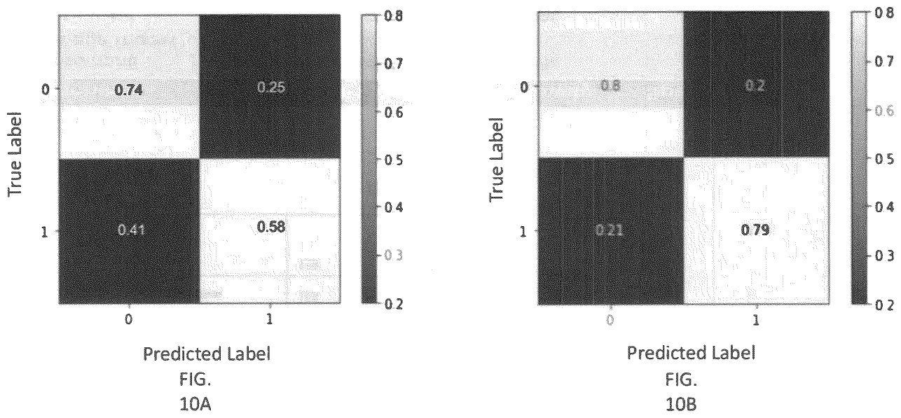

FIG. 9A illustrates the various details of the data subsets used in an embodiment of the present disclosure. The training data subset includes data of 643 patients, among which 448 patients are classified as early DKD patients, and 195 patients are classified as advanced DKD patients. The validation data subset includes data of 168 patients, of which 113 patients are classified as early DKD patients, and 55 patients are classified as advanced DKD patients. The test data subset includes data of 159 patients, among which 125 patients are classified as early DKD patients, and 34 patients are classified as advanced DKD patients.

FIG. 9B illustrates the various parameters of the multilabel image model and the clinical model, according to an example embodiment disclosed herein. The Area Under Curve (AUC) represents the accuracy of the multilabel image model and clinical model, which is 79% and 86% respectively. The F1 score also measures a model's accuracy on a dataset. The F1 score of the multilabel image model and the clinical model is 47% and 63%, respectively. The sensitivity of the model represents its true positive rate (TPR). The sensitivity of the multilabel image model and the clinical model is 58% and 79%, respectively. The specificity of the model represents its true negative rate (TNR). The specificity of the multilabel image model and the clinical model is 74% and 80%.

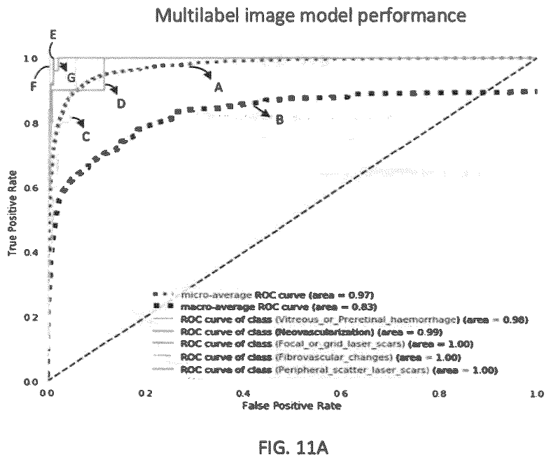

FIG. 10A illustrates a confusion matrix for the multilabel image model, according to an example embodiment disclosed herein. The multilabel image model has an accuracy of 74% for identifying true negatives, and an accuracy of 58% for identifying true positives. FIG. 10B illustrates a confusion matrix for the clinical model, according to an example embodiment disclosed herein. The clinical model has an accuracy of 80% for identifying true negatives, and an accuracy of 79% for identifying true positives.

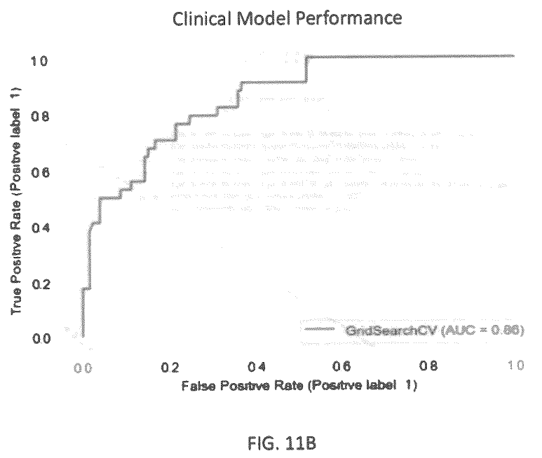

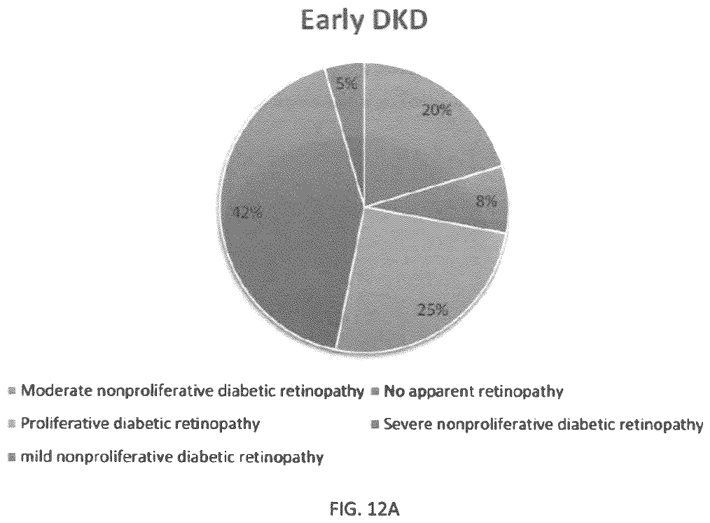

FIG. 11A illustrates a graph depicting the performance of the multilabel image model, according to an example embodiment disclosed herein. The curve denoted by ‘A’ represents the micro-average ROC curve (area=0.97). The curve denoted by ‘B’ represents the macro-average ROC curve (area=0.83). The curve denoted by ‘C’ represents the ROC curve of class vitreous or preretinal hemorrhage (area=0.98). The curve denoted by ‘D’ represents the ROC curve of class neovascularization (area=0.99). The curve denoted by ‘E’ represents the ROC curve of class focal or grid laser scars (area=1.00). The curve denoted by ‘F’ represents the ROC curve of class fibrovascular changes (area=1.00). The curve denoted by ‘G’ represents the ROC curve of class peripheral scatter laser scars (area=1.00).

FIG. 11B illustrates a graph depicting the performance of the clinical model with respect to the true positive rate and the false positive rate, in accordance with an example embodiment of the present disclosure. The AUC is 0.86.

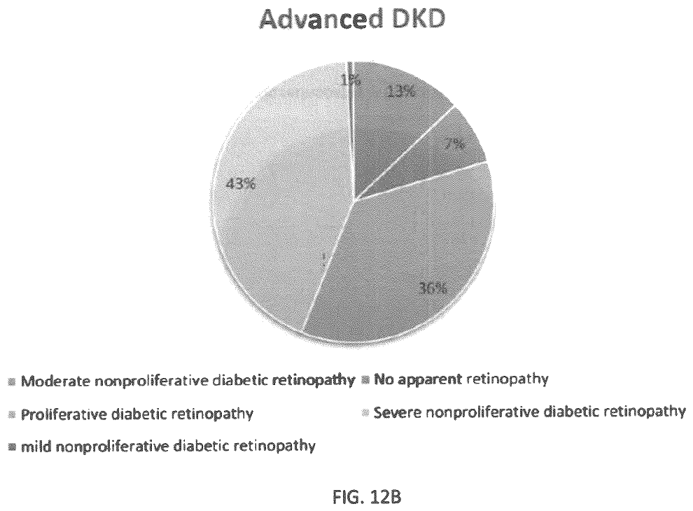

FIG. 12A illustrates a pie chart of the percentage breakdown of DR stages correlating to early stage DKD for a plurality of samples, according to an example embodiment disclosed herein. Out of the early stage DKD cases, 5% had mild non-proliferative DR, 20% had moderate non-proliferative DR, 8% had no apparent retinopathy, 25% had proliferative DR, and 42% had severe non-proliferative DR.

FIG. 12B illustrates a pie chart of the percentage breakdown of DR stages correlating to advanced stage DKD for a plurality of samples, in accordance with an example embodiment of the present disclosure. Out of the advanced stage DKD cases, 1% had mild non-proliferative DR, 13% had moderate non-proliferative DR, 7% had no apparent retinopathy, 36% had proliferative DR, and 43% had severe non-proliferative DR.

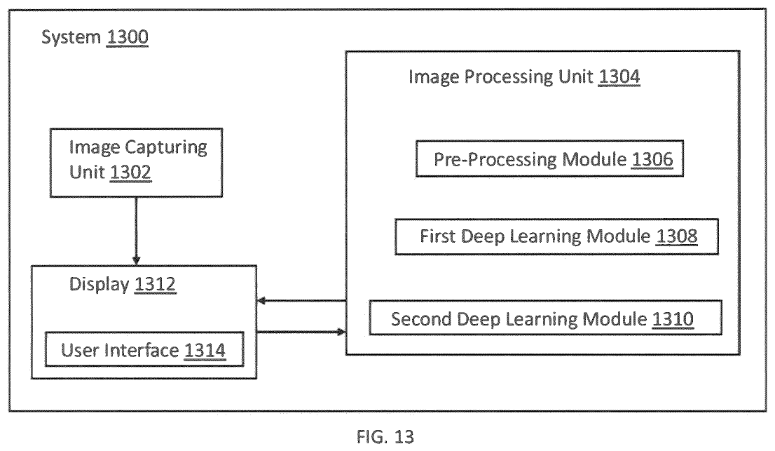

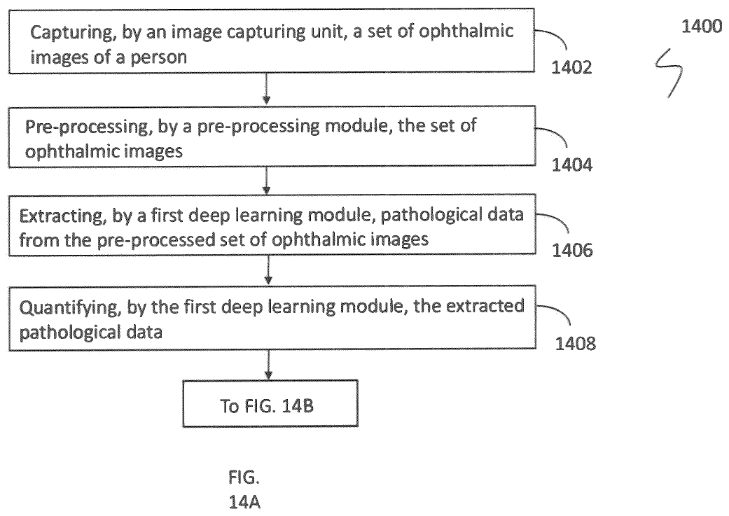

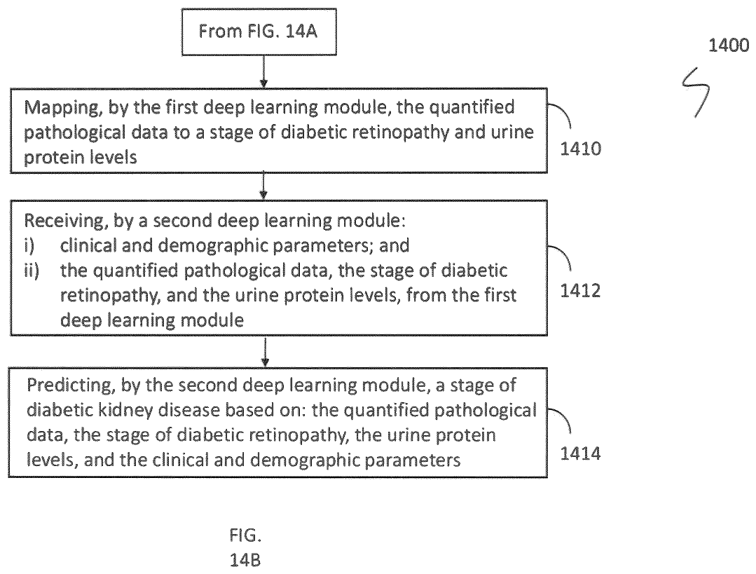

FIG. 13 illustrates a system 1300 for implementing the example embodiments of the present disclosure. The system 1300 comprises an image capturing unit 1302, an image processing unit 1304, and a display 1312.

The image capturing unit 1302 is responsible for capturing a set of ophthalmic images of a person (e.g., a diabetic patient). In one embodiment, the ophthalmic images can be fundus images, and in another embodiment, the ophthalmic images can be OCT images. The image capturing unit 1302 can be a device such as a fundus camera or an OCT machine. The set of ophthalmic images can include images captured of the left and right eye of the diabetic patient.

The set of ophthalmic images are transmitted to the image processing unit 1304. The image processing unit 1304 comprises a pre-processing module 1306, a first deep learning module 1308, and a second deep learning module 1310. The image processing unit 1306 processes the ophthalmic images set obtained/captured by the image capturing unit 1302 to determine whether the ophthalmic images set has the presence of vascular abnormalities patterns for staging DKD.

The pre-processing module 1306 is responsible for pre-processing the ophthalmic images set. In one embodiment, the pre-processing of the captured image involves applying Gaussian blur to smoothen the ophthalmic images set and then applying Ben Graham pre-processing to the smoothened ophthalmic images set. In an embodiment for training the multilabel image model, the pre-processing module 1306 may pre-process a set of reference images (e.g., the training dataset).

The pre-processed ophthalmic images set is then fed to the first deep learning module 1308. The module 1308 generates a first advanced set based on the ophthalmic images set. More particularly, generating the advanced feature set involves extracting vascular abnormalities patterns (i.e., pathological data) from the ophthalmic images set, and quantifying the vascular abnormalities patterns. The module 1308 may employ the multilabel image model for extracting and quantifying the vascular abnormalities patterns. The module 1308 may use a rule parser (e.g., DR stage regex rule parser) for mapping the quantified vascular abnormalities patterns to a stage of DR, which is indicative of the urine protein levels. The first advanced feature set comprises the quantified vascular abnormalities patterns, DR stage, and urine protein levels.

The second deep learning module 1310 generates a second advanced feature set based on the ophthalmic images set, the first advanced feature set, and clinical and demographic data. The second deep learning module 1310 can receive the ophthalmic images set either directly from the image capturing unit 1302 or from the pre-processing module 1306. The second deep learning module 1310 also receives as input the first advanced feature set generated by the first deep learning module 1308 and the clinical and demographic data. The second advanced feature set comprises the stage of diabetic kidney disease.

The display 1312 comprises a user interface 1314 through which the ophthalmic images set can be uploaded and thereby transmitted to the image processing unit 1304. The user interface 1314 can also comprise multiple input fields through which a user can manually input the first advanced feature set and the clinical and demographic data to be utilized by the second deep learning module 1310. The second advanced feature set generated by the second deep learning module 1310 can be displayed on the user interface 1314.

Although not shown in FIG. 13, the system 1300 can comprise at least one memory and at least one processor for carrying out the functionality associated with the image capturing unit 1302, image processing unit 1304, and display 1312. The at least one memory can be a non-transitory computer-readable storage medium that is capable of storing computer program instructions, or computer code, for execution by the at least one processor to result in the performance of method 1400 (as described below). The at least one memory may be multiple memories distributed across multiple computing devices. The at least one memory can include, but is not limited to, random access memory (RAM), read-only memory (ROM), hard drive, solid-state drive, static random access memory (SRAM) etc. The at least one processor may be an electronic component that executes a computer program or computer instructions to result in the functionality of the various constituents of the system 1300. The at least one processor can be a single processor or a plurality of processors. The at least one processor can be a general purpose processor, a Digital Signal Processor (DSP), an Application Specific Integrated Circuit (ASIC), a Field Programmable Gate Array (FPGA) or other programmable logic device, discrete gate or transistor logic, discrete hardware components, or any combination thereof designed to perform the functions described herein. A general purpose processor may be a microprocessor, but in the alternative, the processor may be any conventional processor, controller, microcontroller, or state machine. The at least one processor may also be implemented as a combination of a DSP and a microprocessor, a plurality of microprocessors, one or more microprocessors in conjunction with a DSP core, or any other such configuration.

FIG. 14 illustrates a method 1400 for detecting stage of DKD based on stage of DR, in accordance with an example embodiment of the present disclosure. At step 1402, a set of ophthalmic images of a person are captured, by an image capturing unit 1302. The set of ophthalmic images can correspond to retinal fundus images or OCT images. At step 1404, a pre-processing module 1306 processes the set of ophthalmic images. The pre-processing can involve applying Gaussian blur and Ben Graham pre-processing to smoothen the set of ophthalmic images. In another embodiment, the pre-processing module applies pre-processing techniques to clinical and demographic parameters to eliminate noise in them. At step 1406, a first deep learning module 1308, employing the multilabel image model, extracts pathological data from the set of ophthalmic images. The pathological data is indicative of the vascular abnormalities in the set of ophthalmic images. At step 1408, the first deep learning module 1308 quantifies the extracted pathological data, wherein the quantification is based on the vascular abnormalities in the set of ophthalmic images. At step 1410, the first deep learning module 1308, with the help of the DR stage rule parser, maps the quantified pathological data to a stage of DR. The first deep learning module 1308 also maps the quantified pathological data to urine protein levels. At step 1412, a second deep learning module 1310, employing the clinical model, receives the clinical (e.g., other comorbidities, duration of diabetes, and history of diabetes) and demographic (e.g., age and gender) parameters. In one embodiment, the clinical and demographic parameters may also undergo pre-processing by the pre-processing module 1306. The pre-processing module 1306 may apply Gaussian blur to smooth the clinical and demographic parameters, and eliminate noise from it. The pre-processing module 1306 may also apply Ben Graham pre-processing to the smooth clinical and demographic parameters for de-noising. In addition to the clinical and demographic parameters, the second deep learning module 1310 also receives the output of the first deep learning module 1308, i.e., the quantified pathological data, the stage of DR, and the urine protein levels. Based on the clinical and demographic parameters and the output of the first deep learning module 1308, the second deep learning module 1310 predicts a stage of DKD. The second deep learning module 1310 can also take the pre-processed set of ophthalmic images as an input for predicting the stage of DKD.

In some embodiments, the image processing unit 1304 may divide each image, in the set of ophthalmic images, into four quadrants, wherein based on the vascular abnormalities in each quadrant, the first deep learning module 1308 extracts and quantifies the pathological data in each quadrant. The vascular abnormalities in each quadrant of an ophthalmic image is representative of the vascular abnormalities in the kidney that leads to the leakage of protein. By quantifying the pathological data in each quadrant, the first deep learning module 1308 can map this to the urine protein levels that indicate the extent of protein leakage in the urine.

For the sake of simplicity, the embodiments herein were disclosed with reference to a two-step multilabel image model (employed by the first deep learning module 1308) and a clinical model (employed by the second deep learning module 1310). However, this is not to be construed as limiting, as in some embodiments, the first deep learning module 1308 and the second deep learning module 1310 can employ a plurality of models for performing a respective function. For example, the first deep learning module 1308 can include a first trained deep learning model for extracting and quantifying the pathological data in each quadrant of an ophthalmic image in the set of ophthalmic images. The first deep learning module 1308 can include a second trained deep learning model for mapping (also referred to as “classifying”) the quantified pathological data to the stage of DR. The first deep learning module 1308 can include a third trained deep learning model for mapping the quantified pathological data to urine protein levels. The second deep learning module 1310 can include a fourth trained deep learning model for receiving, from the second and third trained deep learning models, the stage of DR and the urine protein levels, respectively. The fourth trained deep learning model can also predict the stage of DKD based on the inputs received from the second and third trained deep learning models, and the clinical and demographic parameters.

In some embodiments, the method 1400 may comprise further steps not shown and/or may omit certain steps not shown, therefore this should not be construed as limiting the scope of the present disclosure.

In the specification, there has been disclosed exemplary embodiments of the invention. Although specific terms are employed, they are used in a generic and descriptive sense only and not for purposes of limitation of the scope of the invention.

1. A system comprising:
an image capturing unit configured to capture a set of ophthalmic images of a person; and
an image processing unit configured to process the set of ophthalmic images as obtained from the image capturing unit,
wherein the image processing unit comprises:
a pre-processing module configured to pre-process the set of ophthalmic images;
a first deep learning module configured to:
extract pathological data indicative of vascular abnormalities from the pre-processed set of ophthalmic images;
quantify the extracted pathological data based on the vascular abnormalities; and
map the quantified pathological data to a stage of diabetic retinopathy and urine protein levels; and
a second deep learning module configured to:
receive clinical parameters and demographic parameters;
receive, from the first deep leaning module, the quantified pathological data, the stage of diabetic retinopathy, and the urine protein levels; and
predict a stage of diabetic kidney disease based on: the quantified pathological data, the stage of diabetic retinopathy, the urine protein levels, the clinical parameters and the demographic parameters.

2. The system as claimed in claim 1, wherein the vascular abnormalities in the set of ophthalmic images is representative of vascular abnormalities in the kidney that leads to leakage of protein in the urine.

3. The system as claimed in claim 2, wherein the image processing unit is configured to divide each image, in the set of ophthalmic images, into four quadrants, wherein based on the vascular abnormalities in each quadrant, the first deep learning module uses at least one deep learning technique to extract and quantify the pathological data in each quadrant.

4. The system as claimed in claim 3 wherein the first deep learning module maps the quantified pathological data in each quadrant to urine protein levels indicating the extent of protein leakage in the urine.

5. The system as claimed in claim 1, wherein the pre-processing module is configured to:
apply Gaussian blur to perform at least one of the following:
smooth the set of ophthalmic images; or
smooth the clinical and demographic parameters, and eliminate noise in the clinical and demographic parameters; and
apply Ben Graham pre-processing to at least one of the following:
the smooth set of ophthalmic images; or
the smooth clinical and demographic parameters for de-noising.

6. The system as claimed in claim 3, wherein the first deep learning module includes:
a first trained deep learning model for extracting and quantifying the pathological data in each quadrant of an ophthalmic image in the set of ophthalmic images;
a second trained deep learning model for mapping the quantified pathological data to the stage of diabetic retinopathy; and
a third trained deep learning model for mapping the quantified pathological data to the urine protein levels.

7. The system as claimed in claim 6, wherein the second deep learning module includes a fourth trained deep learning model that:
receives, from the second and third trained deep learning models, the stage of diabetic retinopathy and the urine protein levels, respectively; and
predicts the stage of diabetic kidney disease based on the stage of diabetic retinopathy and the urine protein levels.

8. The system as claimed in claim 1, wherein the clinical and demographic parameters include at least one of: age, gender, other comorbidities, duration of diabetes, or history of hypertension.

9. The system as claimed in claim 1, wherein the vascular abnormalities in the set of ophthalmic images is representative of at least one of the following: no abnormalities, microaneurysms, dot hemorrhages, blot hemorrhages, hard exudates, cotton wool spots, intraretinal hemorrhages, venous beading, intraretinal microvascular abnormalities, neovascularization, vitreous hemorrhage or preretinal hemorrhage.

10. The system as claimed in claim 9, wherein the stage of diabetic retinopathy is “no diabetic retinopathy” if the vascular abnormalities patterns are representative of no abnormalities.

11. The system as claimed in claim 9, wherein the stage of diabetic retinopathy is mild non-proliferative diabetic retinopathy if the vascular abnormalities patterns is representative of microaneurysms.

12. The system as claimed in claim 9, wherein the stage of diabetic retinopathy is moderate non-proliferative diabetic retinopathy if the vascular abnormalities patterns is representative of microaneurysms, dot hemorrhages, blot hemorrhages, hard exudates, and cotton wool spots.

13. The system as claimed in claim 9, wherein the stage of diabetic retinopathy is severe non-proliferative diabetic retinopathy if the vascular abnormalities patterns is representative of microaneurysms, dot hemorrhages, blot hemorrhages, hard exudates, cotton wool spots, intraretinal hemorrhages, venous beading, and intraretinal microvascular abnormalities.

14. The system as claimed in claim 9, wherein the stage of diabetic retinopathy is proliferative diabetic retinopathy if the vascular abnormalities patterns is representative of microaneurysms, dot hemorrhages, blot hemorrhages, hard exudates, cotton wool spots, intraretinal hemorrhages, venous beading, intraretinal microvascular abnormalities, neovascularization, vitreous hemorrhage or preretinal hemorrhage.

15. The system as claimed in claim 1, wherein the urine protein levels are categorized as: normal, microalbuminuria, or macroalbuminuria.

16. The system as claimed in claim 1, wherein the predicted stage of diabetic kidney disease is classified as one of: no diabetic kidney disease, early stage diabetic kidney disease, advanced diabetic kidney disease, or late stage diabetic kidney disease.

17. The system as claimed in claim 1, wherein the predicted stage of diabetic kidney disease is indicative of the progression of renal failure, wherein the renal failure is categorized as one of: stable, rapid, or slow.

18. The system as claimed in claim 1, wherein the set of ophthalmic images and the clinical and demographic parameters are respectively used as independent input information to the second deep learning module.

19. The system as claimed in claim 1, wherein the set of ophthalmic images includes fundus images of right and left eyes.

20. The system as claimed in claim 1, wherein the prediction by the second deep learning module is representative of a referable criteria to a nephrologist.

21. The system as claimed in claim 1, comprising a display with a user interface, wherein the quantified pathological data, the stage of diabetic retinopathy, the urine protein levels, and the predicted stage of diabetic kidney disease are displayed on the user interface.

22. The system as claimed in claim 1, wherein the image capturing unit is at least one of: a fundus camera or an optical coherence tomography (OCT) machine.

23. The system as claimed in claim 1, wherein the set of ophthalmic images are infrared images.

24-47. (canceled)
